(19) United States
(12) Patent Application Publication
(10) Pub. No.: US 2002/0110482 A1
(43) Pub. Date: Aug. 15, 2002

(54) Reverse osmosis sanitizer and method therefor

(76) Inventor: Mark Lawton, Las Vegas, NV (US)

Correspondence Address: WEISS & MOY PC, 4204 NORTH BROWN AVENUE, SCOTTSDALE, AZ, 85251, US

(21) Appl. No.: 09/783,172

(22) Filed: Feb. 15, 2001

(51) Int. Cl. A61L 2/23

(52) U.S. Cl. 422/28; 422/282; 62/303; 137/268

(57) ABSTRACT

A reverse osmosis sanitizer and method provide a simple mechanism for periodic maintenance of a reverse osmosis water purification system. The sanitizer comprises a vessel for introducing a cleaning agent such as anti-bacterial hand soap through the water purification system via a detachable connection used to temporarily couple the water supply to the water purification system. The sanitizer and related method clean the membrane, filter housing and storage tank without requiring disassembly of the purification system and without requiring replacement of the membrane. The sanitizer may also be adapted for use in sanitizing an icemaker such as those installed in residential refrigerator/freezers.

### BACKGROUND OF THE INVENTION

1. Field of the Invention

The present invention relates generally to reverse osmosis water purification systems, and more specifically, to a detachable sanitizer for sanitizing a reverse osmosis water purification system.

2. Background of the Invention

Water purification systems are in common use. Recently, reverse osmosis systems have become very popular in residential applications, as the cost of these systems has dropped to make home installation economical. Additionally, industrial uses of reverse osmosis extend to very large systems used in water treatment and desalination plants.

Reverse osmosis water purification systems typically use a perforated membrane manufactured from a polymeric material. Contaminants are removed from a water source by forcing water under slight pressure against the membrane, whereby purified water passes to the other side of the membrane. Water is also directed across the input face of the membrane to remove the contaminants that collect at the input face. This waste water and is typically discarded through a drain-waste system.

Reverse osmosis water purification systems require periodic sanitization in order to properly remove contaminants and to permit maximum water flow to be maintained between sanitizing cycles. Sanitization of a reverse osmosis water purification system is typically performed by disassembling the system and cleaning the storage tank and reverse osmosis membrane. Typically the membrane is replaced at this time. Household bleach is introduced to the storage tank and reverse osmosis filter housing and water supply is restored, permitting the bleach and water to pass through the system, sanitizing the filter housing and storage tank. This method does not clean the membrane, but disposes of it. In addition, the above-described method requires disassembly of the filter housing and storage tank.

Therefore, it would be desirable to provide a reverse osmosis sanitizer and method that clean the membrane and do not require disassembly of the water purification system.

### SUMMARY OF THE INVENTION

The above objective of sanitizing a reverse osmosis water purification system while providing reuse of the membrane and avoiding disassembly of the purification system is accomplished in a reverse osmosis sanitizer and method. The reverse osmosis sanitizer includes a vessel for introducing a cleaning agent that is detachably coupled between the water supply and the reverse osmosis water purification system. The sanitizer may include a coupler for reconnecting water supply hose ends after sanitizing.

The foregoing and other objectives, features, and advantages of the invention will be apparent from the following, more particular, description of the preferred embodiment of the invention, as illustrated in the accompanying drawings.

### BRIEF DESCRIPTION OF THE DRAWINGS

FIG. 1 is a pictorial diagram depicting a reverse osmosis sanitizer in accordance with a preferred embodiment of the present invention.

FIG. 2 is a pictorial diagram depicting inner details of the reverse osmosis sanitizer of FIG. 1.

### DETAILED DESCRIPTION OF THE PREFERRED EMBODIMENTS

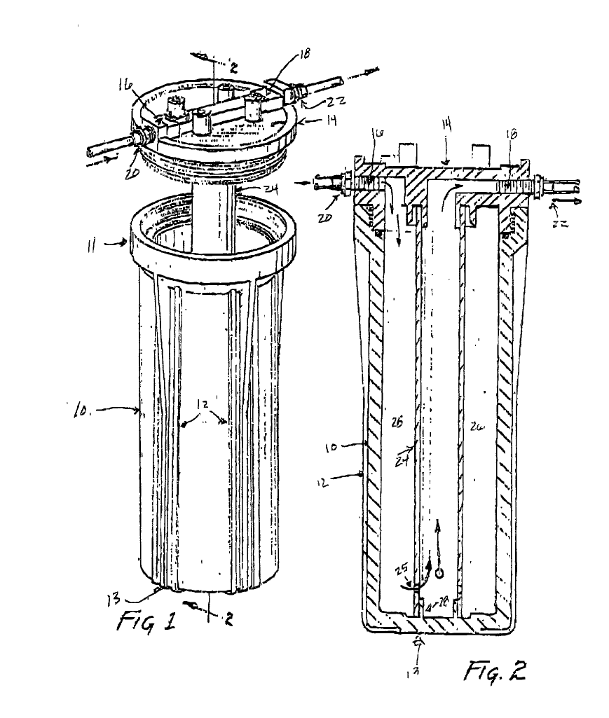

Referring now to the figures and in particular to FIG. 1, a sanitizer for use with a reverse osmosis water purification system is shown. A cylindrical cap 10 has a threaded section on a top collar 11 formed on an outer wall of the cylindrical cap 10 opposite a bottom 13. Ridges 12 are disposed around outer wall of cylindrical cap 10, to aid in removal of cylindrical cap 10 from a port 14. Port 14 has a water supply inlet 16 and a sanitizing mixture outlet 18. Water supply inlet 16 can be detachably coupled to a water supply line via a second coupler 20 that is threaded into inlet 16 which can be made by threading a section in the body of port 14. Sanitizing mixture outlet 18 can be detachably coupled to a reverse osmosis water purification system via a second coupler 22 that is threaded into outlet 18 which can be made by threading a second section in the body of port 14. First coupler 20 and second coupler 22 each have a threaded end for attachment to the sanitizing system and a slip-fit end for detachably coupling to flexible hose.

When sanitizing a residential reverse osmosis water purification system for the first time, the flexible water supply hose, which is generally plastic ¼″ hose can be cut with a razor blade. The cut end extending from the water supply can be slipped over the barbed end of first coupler 20 and the second cut end that extends to the water purification system can be slipped into the end of second coupler 22. A shunt coupler having a slip fitting on each end may be supplied with the sanitizer for reconnecting the cut hose ends after sanitizing the water purification system and to facilitate subsequent use of the sanitizer.

Referring now to FIG. 2 which is a cross-section or FIG. 1, the internal details of the reverse osmosis sanitizer are shown. A distribution pipe 24 extends from port 14 to the bottom of cylindrical cap 10. Distribution pipe 24 is affixed to port 14 and is coupled to outlet 18. The end of distribution pipe 24 is held by an annulus 28 that is sized for a glued connection (using, for example, pvc cement) to the inside diameter of distribution pipe 24. One or more perforations 25 in distribution pipe 24 provide flow from a chamber 26 that is formed by the inside wall of cylindrical cap 10 and the outside of distribution pipe 24. Perforations 25 are sized to control the flow of liquid from chamber 26 to the inside of distribution pipe 24 and from distribution pipe 24 out through outlet 18. The flow is controlled in order to control mixing of a cleaning agent such as anti-bacterial hand soap, which is introduced to chamber 26 and the inside of distribution pipe 24 by pouring it into cylindrical cap 10 prior to screwing cylindrical cap 10 onto port 14. When a water supply connected to coupler 20 is activated, water enters chamber 26 and mixes with the cleaning agent. The mixture exits the sanitizer via coupler 22 and enters the water purification system.

The sanitizer is used by preparing the reverse osmosis water purification system as for a filter change. The water supply is turned off via a valve. The storage tank is drained and then restored to its proper pressure (generally 6 to 10 psi). Cap 10 is unscrewed from the sanitizer and a cleaning agent such as non-toxic unscented anti-bacterial hand soap is poured into cap 10 (generally 2-3 ounces for residential units). The cap is then reattached to port 14. Then, the water supply is disconnected from the water purification system and coupled to coupler 20. Next, the water purification system is coupled to the water purification system via coupler 22 and the water supply is turned on, introducing cleaning agent mixed with water throughout the water purification system. After two hours, or after the storage tank has filled to a predetermined level, the water supply is again turned off via a valve. After waiting a suitable time for pressure to be relieved via flow through the purification system, the sanitizer is decoupled from the water purification system and water supply and the water supply is reconnected to the water purification system. At this point, all parts of the water purification system are now sanitized, including the membrane and the storage tank. The storage tank is then emptied (discarding the cleaning agent mixture) and permitted to refill. After the tank fills again, the water should again be discarded. The next tank of water will be suitable for use.

The above-described embodiment is directed to a sanitizer adapter for a residential reverse osmosis water purification system, but fittings and/or appropriate-sized vessels may be fabricated to accommodate other applications. For example, ¾″ fittings and a larger vessel (to mix a larger volume of cleaning agent) can be used to adapt the above-described embodiment to an industrial application. Calcium deposits (hardness) may also be removed from industrial units by using a brine solution rather than the cleaning agent with little degradation in the quality of the membrane filter characteristics. Water softener failures often cause lime deposits and other hardness buildup on the reverse osmosis system membranes.

The above-described embodiment may also be used with different fittings adapted to sanitize a residential icemaker and associated filters, such as those found installed within refrigerator/freezers. Less sanitizing agent is generally required for this application, so the vessel depicted is suitable for this use as well as for sanitizing the main water purification system. Rather than discarding sanitizing water, a quantity of ice must be discarded.

While the invention has been particularly shown and described with reference to the preferred embodiments thereof, it will be understood by those skilled in the art that the foregoing and other changes in form, and details may be made therein without departing from the spirit and scope of the invention.

What is claimed is:

1. A sanitizer for sanitizing a residential reverse osmosis water purification system, said sanitizer comprising:
a first coupler for coupling said sanitizer system to a water supply;
a vessel having an inlet coupled to said first coupler for mixing a cleaning agent with water from said water supply; and
a second coupler coupled to an outlet of said vessel, for coupling said sanitizer to said water purification system, whereby said cleaning agent may be introduced through said water purification system.

2. The sanitizer of claim 1, further comprising a shunt for detachably re-coupling said water supply to said water purification system when said sanitizer is detached from said water supply and said water purification system.

3. The sanitizer of claim 1, wherein said vessel comprises:
a port including said inlet and said outlet;
a cylindrical cap having a bottom end and a wall, said wall having a threaded section opposite said bottom for detachably coupling said cap to said port; and
a distribution tube affixed to said port, coupled to said outlet, and disposed axially within said cylindrical cap when said cylindrical cap is coupled to said port, and wherein said distribution tube is perforated for controlling flow from a chamber formed between an inner side of said wall of said cylindrical cap and the outside surface of said distribution tube to the inside of said distribution tube.

4. The sanitizer of claim 3, wherein said inlet is a one-quarter inch threaded section formed through said port and said outlet is a one-quarter inch threaded section formed through said port.

5. The sanitizer of claim 4, wherein said first coupler has a threaded end for threading into said inlet and a slip-fit end for detachably coupling a flexible hose from said water supply.

6. The sanitizer of claim 5, wherein said second coupler has a threaded end for threading into said outlet and a slip-fit end for detachably coupling a flexible hose to said water purification system.

7. The sanitizer of claim 6, further comprising a shunt for detachably re-coupling said water supply to said water purification system when said sanitizer is detached from said water supply and said water purification system, wherein said shunt has a first slip-fit end for detachably coupling a flexible hose from said water supply and a second slip-fit end for detachably coupling a flexible hose to said water purification system.

8. The sanitizer of claim 3, wherein said cap includes an annulus formed on an inner side of said bottom for supporting said distribution tube.

9. The sanitizer of claim 8, wherein a diameter of said annulus is slightly smaller than an inside diameter of said distribution tube for providing a friction fit between said distribution tube and said cap.

10. The sanitizer of claim 1, wherein said cap includes ridges disposed on an exterior of said wall for adapting said cap to a filter wrench, whereby said cap may be removed from said port.

11. The sanitizer of claim 1, wherein said first coupler and said second coupler are adapted for attachment to a rigid pipe system for industrial applications.

12. The sanitizer of claim 1, further comprising a third coupler and a fourth coupler each having a first end adapted for attachment to said port and a second end adapted for attachment inline with an ice maker water supply.

13. A method for sanitizing a reverse osmosis water purification system, said method comprising:
disconnecting a water supply from a supply connection of said water purification system;
adding a cleaning agent to a sanitizer external to said water purification system;
connecting a water supply to said sanitizer;
connecting said sanitizer to said supply connection of said water purification system; and
introducing water to said sanitizer so that said cleaning agent is passed through said water purification system.

14. The method of claim 13, wherein said cleaning agent is anti-bacterial hand soap.

15. The method of claim 13, wherein said adding is performed by:
unscrewing a cap from said sanitizer;
pouring said cleaning agent into said cap; and
replacing said cap on said sanitizer.

16. The method of claim 13, further comprising mixing said cleaning agent with water from said water supply via a perforated distribution tube.

17. The method of claim 11, further comprising reconnecting said water supply to said supply connection via a shunt.

18. A method for sanitizing an icemaker water supply, said method comprising:
disconnecting a water supply from a supply connection of said icemaker;
adding a cleaning agent to a sanitizer external to said icemaker;
connecting a water supply to said sanitizer;
connecting said sanitizer to said supply connection of said icemaker; and
introducing water to said sanitizer so that said cleaning agent is passed through said icemaker.

19. The method of claim 18, further comprising mixing said cleaning agent with water from said water supply via a perforated distribution tube.

20. The method of claim 18, further comprising reconnecting said water supply to said supply connection via a shunt.
